SEQUENCE LISTING (2 sequences)

SEQ ID NO: 1 - DNA, 5561 bp, Homo sapiens
aaagcagcagagacgctgcagagggcttttcttagacatcaactgcagacggctggcaggatagaagcagcggctcacttggactttttcaccagggaaatcagagacaatgatggggctcttccccagaactacaggggctctggccatcttcgtggtggtcatattggttcatggagaattgcgaatagagactaaaggtcaatatgatgaagaagagatgactatgcaacaagctaaaagaaggcaaaaacgtgaatgggtgaaatttgccaaaccctgcagagaaggagaagataactcaaaaagaaacccaattgccaagattacttcagattaccaagcaacccagaaaatcacctaccgaatctctggagtgggaatcgatcagccgccttttggaatctttgttgttgacaaaaacactggagatattaacataacagctatagtcgaccgggaggaaactccaagcttcctgatcacatgtcgggctctaaatgcccaaggactagatgtagagaaaccacttatactaacggttaaaattttggatattaatgataatcctccagtattttcacaacaaattttcatgggtgaaattgaagaaaatagtgcctcaaactcactggtgatgatactaaatgccacagatgcagatgaaccaaaccacttgaattctaaaattgccttcaaaattgtctctcaggaaccagcaggcacacccatgttcctcctaagcagaaacactggggaagtccgtactttgaccaattctcttgaccgagagcaagctagcagctatcgtctggttgtgagtggtgcagacaaagatggagaaggactatcaactcaatgtgaatgtaatattaaagtgaaagatgtcaacgataacttcccaatgtttagagactctcagtattcagcacgtattgaagaaaatattttaagttctgaattacttcgatttcaagtaacagatttggatgaagagtacacagataattggcttgcagtatatttctttacctctgggaatgaaggaaattggtttgaaatacaaactgatcctagaactaatgaaggcatcctgaaagtggtgaaggctctagattatgaacaactacaaagcgtgaaacttagtattgctgtcaaaaacaaagctgaatttcaccaatcagttatctctcgataccgagttcagtcaaccccagtcacaattcaggtaataaatgtaagagaaggaattgcattccgtcctgcttccaagacatttactgtgcaaaaaggcataagtagcaaaaaattggtggattatatcctgggaacatatcaagccatcgatgaggacactaacaaagctgcctcaaatgtcaaatatgtcatgggacgtaacgatggtggatacctaatgattgattcaaaaactgctgaaatcaaatttgtcaaaaatatgaaccgagattctactttcatagttaacaaaacaatcacagctgaggttctggccatagatgaatacacgggtaaaacttctacaggcacggtatatgttagagtacccgatttcaatgacaattgtccaacagctgtcctcgaaaaagatgcagtttgcagttcttcaccttccgtggttgtctccgctagaacactgaataatagatacactggcccctatacatttgcactggaagatcaacctgtaaagttgcctgccgtatggagtatcacaaccctcaatgctacctcggccctcctcagagcccaggaacagatacctcctggagtataccacatctccctggtacttacagacagtcagaacaatcggtgtgagatgccacgcagcttgacactggaagtctgtcagtgtgacaacaggggcatctgtggaacttcttacccaaccacaagccctgggaccaggtatggcaggccgcactcagggaggctggggcctgccgccatcggcctgctgctccttggtctcctgctgctgctgttggccccccttctgctgttgacctgtgactgtggggcaggttctactgggggagtgacaggtggttttatcccagttcctgatggctcagaaggaacaattcatcagtggggaattgaaggagcccatcctgaagacaaggaaatcacaaatatttgtgtgcctcctgtaacagccaatggagccgatttcatggaaagttctgaagtttgtacaaatacgtatgccagaggcacagcggtggaaggcacttcaggaatggaaatgaccactaagcttggagcagccactgaatctggaggtgctgcaggctttgcaacagggacagtgtcaggagctgcttcaggattcggagcagccactggagttggcatctgttcctcagggcagtctggaaccatgagaacaaggcattccactggaggaaccaataaggactacgctgatggggcgataagcatgaattttctggactcctacttttctcagaaagcatttgcctgtgcggaggaagacgatggccaggaagcaaatgactgcttgttgatctatgataatgaaggcgcagatgccactggttctcctgtgggctccgtgggttgttgcagttttattgctgatgacctggatgacagcttcttggactcacttggacccaaatttaaaaaacttgcagagataagccttggtgttgatggtgaaggcaaagaagttcagccaccctctaaagacagcggttatgggattgaatcctgtggccatcccatagaagtccagcagacaggatttgttaagtgccagactttgtcaggaagtcaaggagcttctgctttgtccacctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtactattcaaattgtagtaaatcttaaagtttttcaaaaccctaaaatcatattcgccaggaaattttcctaaacattcttaagcttctatttttcccctgccaaaggaaggtgtttatcattttaaaatgcaatgtgatttagtggattaagcaggagcgctggttcttgtctccattgccttttcttatatcattgataatgatgtaagaatcacaaggggccgggcgcggtggctcacgcctgtaatcccagcactttgggaggccgaggcaggtggatcatgaggtcaggagatcgagaccatcctggctaacaaggtgaaaccccgtctctactaaaaatacaaaaaattagccgggcgcagtggcgggcgcctgtagtcccagctactcgggaggctgaggcaggagaatggcatgaacccgggaagcggagcttgcagtgagccgagattgcgccactgcagtccgcagtccggcctgggcgacagagcgagactccgtctcaaaaaaaaaaaaaaaaaaagaatcacaaggtatttgctaaagcattttgagctgcttggaaaaagggaagtagttgcagtagagtttcttccatcttcttggtgctgggaagccatatatgtgtcttttactcaagctaaggggtataagcttatgtgttgaatttgctacatctatatttcacatattctcacaataagagaattttgaaatagaaatatcatagaacatttaagaaagtttagtataaataatattttgtgtgttttaatccctttgaagggatctatccaaagaaaatattttacactgagctccttcctacacgtctcagtaacagatcctgtgttagtctttgaaaatagctcattttttaaatgtcagtgagtagatgtagcatacatatgatgtataatgacgtgtattatgttaacaatgtctgcagattttgtaggaatacaaaacatggccttttttataagcaaaacgggccaatgactagaataacacatagggcaatctgtgaatatgtattataagcagcattccagaaaagtagttggtgaaataattttcaagtcaaaaagggatatggaaagggaattatgagtaacctctattttttaagccttgcttttaaattaaacagctacagccatttaagccttgaggataataaagcttgagagtaataatgttaggttagcaaaggtttagatgtatcacttcatgcatgctaccatgatagtaatgcagctcttcgagtcatttctggtcattcaagatattcacccttttgcccatagaaagcaccctacctcacctgcttactgacattgtcttagctgatcacaagatcattatcagcctccattattccttactgtatataaaatacagagttttatattttcctttcttcgtttttcaccatattcaaaacctaaatttgtttttgcagatggaatgcaaagtaatcaagtgtttgtgctttcacctagaagggtgtggtcctgaaggaaagaggtcccctaaatatcccccaccctggtgctcctccctctccctggtaccctgactaccaggaagtcaggtgctagagcagctggagaagtgcaggcagcctgtgcttccacagatgggggtgctgctgcaacaaggctttcaatgtgcccatcttaggtgggagaagctagatcctgtgcagcagcctggtaagtcctgaggaggttccattgctcttcctgctgctgtcctttgcttctcaacggtggctcgctctacagtctagagcacatgcagctaacttgtgcctctgcttatgcatgagggttaaattaacaaccataaccttcatttgaagttcaaaggtgtattcaggatcctcaaagcattttaaccttgccgcttaaaacccaatttaccgtgaaatgggaattttgctgcattgttaaactgtagtggaaaccatgctatagtaataaaggttatataagagagaaattgaaattaaatgtgtttttaaatttcaaaaaaaaatcaatctttaggatgacttaaaaattgatttgccatgtaaaatgtatctgcattttttacacaaaacttgttttaagcataaaattttaaaactgtactacttgatgtattatacattttgaaccatatgtattaaaccataaacagtataatgttgttataataaaacaggcaataaatttataaataaaagctgaaaaaaaaaaaa

SEQ ID NO: 2 - protein, 999 aa, Homo sapiens
MMGLFPRTTGALAIFVVVILVHGELRIETKGQYDEEEMTMQQAKRRQKREWVKFAKPCREGEDNSKRNPIAKITSDYQATQKITYRISGVGIDQPPFGIFVVDKNTGDINITAIVDREETPSFLITCRALNAQGLDVEKPLILTVKILDINDNPPVFSQQIFMGEIEENSASNSLVMILNATDADEPNHLNSKIAFKIVSQEPAGTPMFLLSRNTGEVRTLTNSLDREQASSYRLVVSGADKDGEGLSTQCECNIKVKDVNDNFPMFRDSQYSARIEENILSSELLRFQVTDLDEEYTDNWLAVYFFTSGNEGNWFEIQTDPRTNEGILKVVKALDYEQLQSVKLSIAVKNKAEFHQSVISRYRVQSTPVTIQVINVREGIAFRPASKTFTVQKGISSKKLVDYILGTYQAIDEDTNKAASNVKYVMGRNDGGYLMIDSKTAEIKFVKNMNRDSTFIVNKTITAEVLAIDEYTGKTSTGTVYVRVPDFNDNCPTAVLEKDAVCSSSPSVVVSARTLNNRYTGPYTFALEDQPVKLPAVWSITTLNATSALLRAQEQIPPGVYHISLVLTDSQNNRCEMPRSLTLEVCQCDNRGICGTSYPTTSPGTRYGRPHSGRLGPAAIGLLLLGLLLLLLAPLLLLTCDCGAGSTGGVTGGFIPVPDGSEGTIHQWGIEGAHPEDKEITNICVPPVTANGADFMESSEVCTNTYARGTAVEGTSGMEMTTKLGAATESGGAAGFATGTVSGAASGFGAATGVGICSSGQSGTMRTRHSTGGTNKDYADGAISMNFLDSYFSQKAFACAEEDDGQEANDCLLIYDNEGADATGSPVGSVGCCSFIADDLDDSFLDSLGPKFKKLAEISLGVDGEGKEVQPPSKDSGYGIESCGHPIEVQQTGFVKCQTLSGSQGASALSTSGSVQPAVSIPDPLQHGNYLVTETYSASGSLVQPSTAGFDPLLTQNVIVTERVICPISSVPGNLAGPTQLRGSHTMLCTEDPCSRLI

(19) United States
(12) Patent Application Publication
(10) Pub. No.: US 2018/0321248 A1
(43) Pub. Date: Nov. 8, 2018

(54) DEVELOPMENT OF PROGNOSTIC MARKERS DSG-3 FROM THE SALIVA OF ORAL CANCER PATIENTS

(71) Applicant: University of Southern California, Los Angeles, CA (US)

(72) Inventors: Uttam K. SINHA, Los Angeles, CA (US); Rizwan MASOOD, Walnut, CA (US)

(21) Appl. No.: 15/773,147

(22) PCT Filed: Nov. 4, 2016
(86) PCT No.: PCT/US2016/060705

§ 371 Date: May 2, 2018

Related U.S. Application Data
(60) Provisional application No. 62/252,338, filed on Nov. 6, 2015.

(51) Int. Cl. G01N 33/574; G01N 33/68; G01N 33/50

(52) U.S. Cl. CPC G01N 33/57488; G01N 33/6869; C12Q 1/6886; G01N 33/5091; G01N 33/6866

(57) ABSTRACT

A method for the early detection of head and neck cancer is provided. The method includes obtaining a saliva sample from a subject, determining the expression level of Desmoglien 3 (DSG3) in the saliva sample, and detecting head and neck cancer by the expression level of DSG3.

### RELATED APPLICATIONS

This application claims the benefit of U.S. Provisional Application No. 62/252,338, filed Nov. 6, 2015, the entire contents of which are incorporated herein by reference in its entirety.

### FIELD OF THE INVENTION

The present invention relates to early cancer detection methods.

### BACKGROUND OF THE INVENTION

With more than half a million new cases annually, head and neck squamous cell carcinoma (HNSCC) is the sixth most common cancer worldwide. In 2010, it accounted for 49,260 new cancer diagnoses and 11,480 deaths in the United States. Prognosis of HNSCC is largely determined by the extent of local invasion and metastasis to the cervical lymph nodes at diagnosis. Nearly 50% of patients with HNSCC have lymphatic metastasis, which accounts for the persistently poor prognosis over the past several decades despite advancement in our understanding of prevention and treatment. Biomarkers may provide new pathways for early cancer detection. Although biomarkers are still limited in their insufficient diagnostic sensitivity and specificity, saliva has the potential to be an important tool in early detection within the head and neck squamous cell carcinoma (HNSCC) patient population. In this population, cancer usually initiates in the oral cavity and oropharynx, where saliva is always in contact with the tumor site—hence, the need to investigate saliva as a potential source for detection of biomarkers in HNSCC. Early detection is a key factor in the management of HNSCC and improvement of survival rate of this patient population. The HNSCC patient population seems an ideal demographic within which identification of salivary biomarkers may have tremendous potential for detection of early stage cancer.

Squamous cell carcinoma arises from the squamous lining of the mucosal surfaces of the upper aerodigestive tract, including the oral cavity, pharynx, larynx, and sinonasal tract. Head and neck squamous cell carcinoma (HNSCC) comprises more than 90% of cancers of the head and neck region. Clinical staging of HNSCC is done based on tumor size, metastasis to the cervical lymph nodes (regional metastasis), and metastasis to the distant organ (e.g., lungs). Typically, this cancer metastasizes to the cervical lymph nodes and distant metastasis is very uncommon. Depending on the clinical presentation, HNSCC is classified from Stage I to Stage IV. Treatment is quite morbid and results in significant functional as well as aesthetic deficits, such as impairment of speech and swallowing and facial deformity. Treatment failure and locoregional recurrence (primary site and cervical lymph nodes) are common and occur in up to 50% of patients and account for the majority of deaths (FIG. 1). A large number of these patients presents at advanced stage (Stage III or IV) and only 50%-60% of patients are alive at 5 years after diagnosis. The high rate of local recurrence produced the long-held notion of a “condemned tissue” composed of the cancerous cells, adjacent epithelial, stromal, and immune cells and their surrounding matrix in the upper aerodigestive tract.

The extracellular matrix (ECM) is a collection of extracellular molecules secreted by cells that provides structural and biochemical support to the surrounding cells. Cell adhesion, cell-to-cell communication and differentiation are common functions of the ECM. Cadherins (named for “calcium-dependent adhesion”) are a class of type-1 transmembrane proteins (FIG. 2). They play important roles in cell adhesion, forming adherens junctions to bind cells within tissues together. The cadherin superfamily includes cadherins, protocadherins, desmogleins, and desmocollins, and more. Desmoglein 3 (FIG. 3) is a member of the cadherin cell adhesion molecule superfamily.

Most currently used markers have been identified in head and neck squamous cell carcinoma (HNSCC) cancer cell lines or in biopsy specimens from advanced stage cancers. The invasive natures of a biopsy make it unsuitable for cancer-screening in high-risk populations. Consequently, there is a need for the development of new diagnostic tools in improving early detection.

### SUMMARY OF THE INVENTION

One object of the present invention is to provide a method for the early detection of head and neck cancer. The method includes the steps of obtaining a saliva sample from a subject, determining the expression level of Desmoglien 3 (DSG3) in the saliva sample, and detecting head and neck cancer by the expression level of DSG3.

According to one embodiment, the step of detecting head and neck cancer includes comparing the expression level of DSG3 with the average expression levels of DSG3 from control saliva samples and advanced stage cancer saliva samples. The control samples are from subjects that can include a) smokers, b) drinkers, c) smokers and drinkers, and d) non-smokers and non-drinkers. All control subjects had not yet developed head and neck cancer. A determination that the level of expression of DSG3 in the saliva sample is greater than the average level of expression of DSG3 in the control saliva samples, but is less than the average DSG3 expression level of the advanced stage cancer saliva samples is indicative of the early detection of head and neck cancer in the subject.

According to another embodiment, the head and neck cancer is lip, oral cavity, tongue, throat, nasal cavity, paranasal sinuses, pharynx, larynx, or salivary glands and cervical lymph nodes of the neck.

According to another embodiment, the DSG3 expression levels of the saliva samples are at least two times greater than the DSG3 expression levels of the control saliva samples.

According to another embodiment, the DSG3 expression levels of the saliva samples are at least five times greater than the DSG3 expression levels of the control saliva samples.

Another object of the present invention is to treat head and neck cancer by performing one or more procedures that include radiation therapy, surgery and chemotherapy on a subject. The procedure is performed when early detection of head and neck cancer is detected by a method that includes the steps of obtaining a saliva sample from the subject, determining the expression level of Desmoglien 3 (DSG3) in the saliva sample, and detecting head and neck cancer by the expression level of DSG3. The step of detecting head and neck cancer includes comparing the expression level of DSG3 with the average expression levels of DSG3 from control saliva samples and advanced stage cancer saliva samples. The control samples are from subjects that can include a) smokers, b) drinkers, c) smokers and drinkers, and d) non-smokers and non-drinkers. All control subjects had not yet developed head and neck cancer. A determination that the level of expression of DSG3 in the saliva sample is greater than the average level of expression of DSG3 in the control saliva samples, but is less than the average DSG3 expression level of the advanced stage cancer saliva samples is indicative of the early detection of head and neck cancer in the subject.

Another object of the present invention to provide a method for the early detection of head and neck cancer that includes the steps of obtaining a saliva sample from a subject and determining expression levels of one or more other biomarkers in addition to DSG3. Head and neck cancer is detected by the expression level of DSG3 and the one or more other biomarkers. These biomarkers can include IL-1, IL-6, IL-8, VEGF, TGFβ, TNFα, MMP-7, plasminogen activated (PA), uPA, IGF, and INF-2 proteins.

According to one embodiment, the expression levels of DSG3 and one or more of these biomarkers are then compared to control and advanced stage cancer saliva samples. A determination that the level of expression of DSG3 and the one or more of the other biomarkers in the saliva sample is greater than the average level of expression of DSG3 and the one or more of the other biomarkers in the control saliva samples, but is less than the average DSG3 and the one or more of the other biomarkers expression level of the advanced stage cancer saliva samples is indicative of the early detection of head and neck cancer in the subject.

According to another embodiment, the one or more other biomarkers include IL-8.

According to another embodiment, the DSG3 and IL-8 expression levels of the saliva samples are at least two times greater than the DSG3 and IL-8 expression levels of the control saliva samples.

According to another embodiment, the DSG3 and IL-8 expression levels of the saliva samples are at least five times greater than the DSG3 and IL-8 expression levels of the control saliva samples.

Another object of the present invention is to treat head and neck cancer by performing one or more procedures that include radiation therapy, surgery and chemotherapy on a subject. The procedure is performed when early detection of head and neck cancer is detected by a method that includes the steps of obtaining a saliva sample from the subject and determining expression levels of one or more other biomarkers in addition to DSG3. Head and neck cancer is detected by the expression level of DSG3 and the one or more other biomarkers. These biomarkers can include IL-1, IL-6, IL-8, VEGF, TGFβ, TNFα, MMP-7, plasminogen activated (PA), uPA, IGF, and INF-2 proteins. The expression levels of these biomarkers are compared to control and advanced stage cancer saliva samples. A determination that the level of expression of DSG3 and the one or more of the other biomarkers in the saliva sample is greater than the average level of expression of DSG3 and the one or more of the other biomarkers in the control saliva samples, but is less than the average DSG3 and the one or more of the other biomarkers expression level of the advanced stage cancer saliva samples is indicative of the early detection of head and neck cancer in the subject.

Other aspects and advantages of the invention will be apparent from the following description and the appended claims.

### BRIEF DESCRIPTION OF THE DRAWINGS

FIG. 1: Recurrent HNSCC of the oral cavity involving the cheek skin.

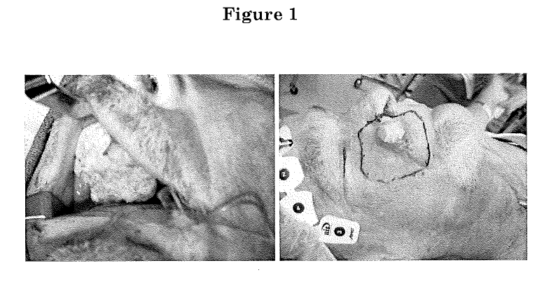

FIG. 2: Right panel: Cadherin superfamily proteins bind the cell membranes and are connected with intracellular proteins through cytoplasmic anchoring plaques to control cellular functions. Left panel: Electron microscopic pictures of these anchoring proteins of 2 adjacent cells.

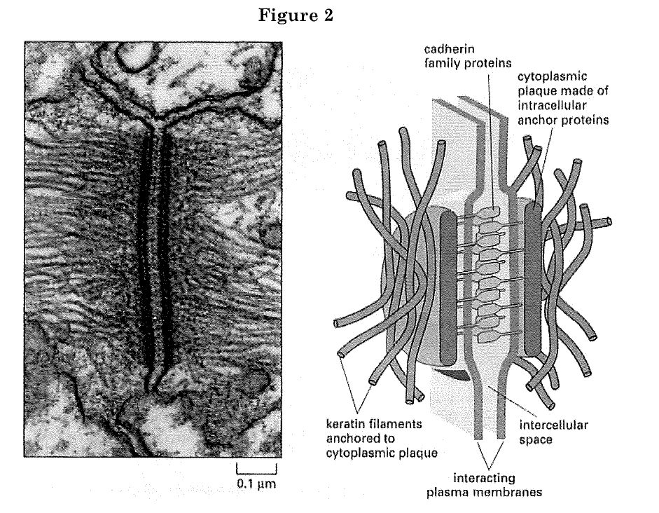

FIG. 3: DSG1 and DSG3, members of cadherins superfamily tightly bind the epithelial cells together.

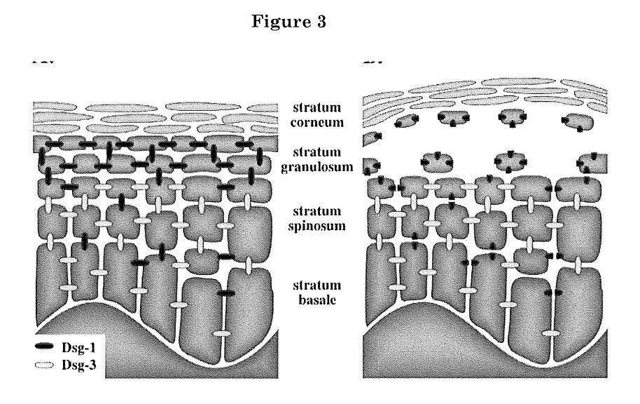

FIG. 4: Expression of DSG-3 from several controls, early stage and advanced stage of oral cancer.

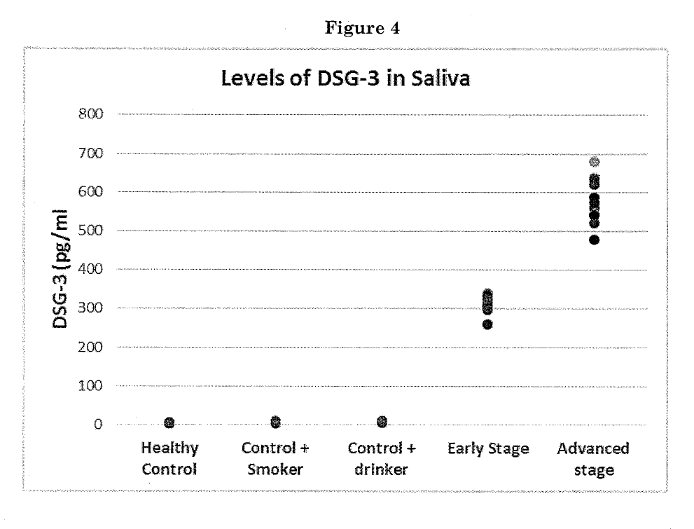

FIG. 5: DSG-3 levels in the saliva of healthy controls and with risk factors.

FIG. 6: DSG-3 expression in the saliva of healthy controls and cancerous patients.

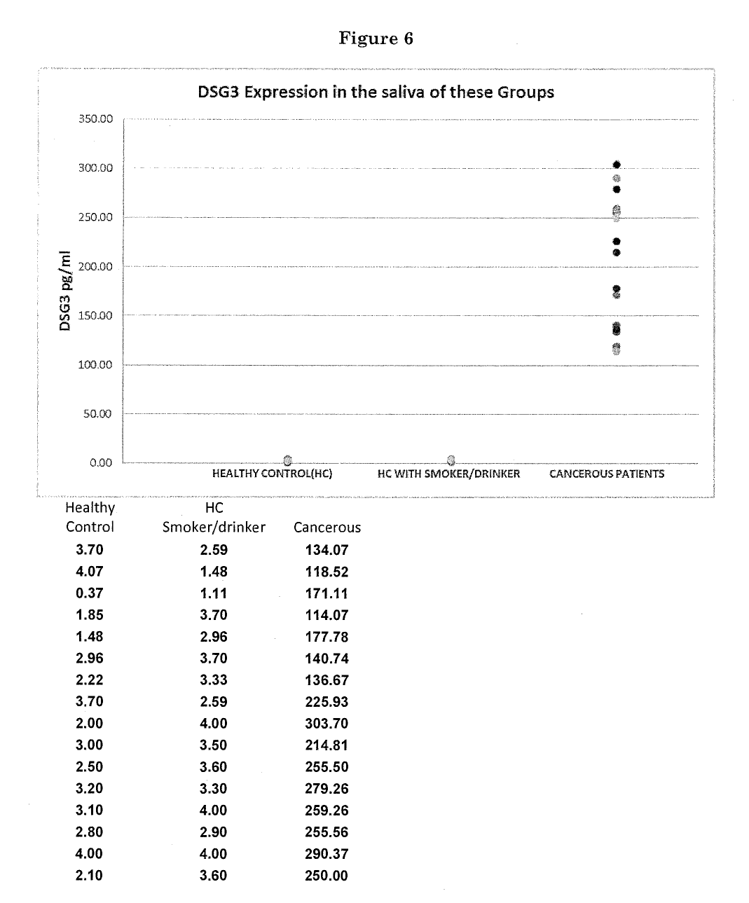

FIG. 7: DSG-3 expression in the saliva of pre-cancerous group vs cancerous group.

FIG. 8: DSG-3 levels in the saliva of cancerous patients with or without lymph node involvement.

### DETAILED DESCRIPTION OF THE INVENTION

A “biomarker” as used herein refers to a molecular indicator that is associated with a particular pathological or physiological state. The “biomarker” as used herein is a molecular indicator for cancer, more specifically an indicator for head and neck cancer.

As used herein the term “cancer” refers to or describes the physiological condition in mammals that is typically characterized by abnormal and uncontrolled cell division or cell growth. Examples of cancer include but are not limited to, carcinoma, lymphoma, blastoma, sarcoma, and leukemia. More specific examples of such cancers include breast, brain, bladder, prostate, colon, intestinal, squamous cell, lung, stomach, pancreatic, cervical, ovarian, liver, skin, colorectal, endometrial, salivary gland, kidney, thyroid, various types of head and neck cancer, and the like. More specifically, “head and neck cancer” refers to any cancer in the head or neck region of the body. Most head and neck cancers are squamous cell carcinomas, but some may be exophilic or endophilic. Examples of head and neck cancers include but are not limited to the lip, oral cavity (mouth), tongue, throat, nasal cavity, paranasal sinuses, pharynx, larynx, salivary glands and cervical lymph nodes of the neck, and the like.

As used herein, a “subject” is preferably a human, non-human primate, cow, horse, pig, sheep, goat, dog, cat, or rodent. In all embodiments, human subjects are preferred. The “subject” may be at risk of developing head and neck cancer, may be suspected of having head and neck, or may have head and neck cancer.

As used herein, the phrase “treating head and neck cancer” means to have one or more of the following effects: to inhibit the formation or spread of primary tumors, macrometastases or micrometastases, or decrease the size of primary tumors, macrometastases or micrometastases.

As used herein, the level of expression of biomarkers can be used for the early diagnoses of cancer in a subject. In these determinations, the level of expression of the biomarker is diagnostic of cancer if the level of expression is above a control level determined for that biopsy type, but below the biomarker expression level for advanced stage cancer biopsies. The control level of expression can be determined using standard methods known to those of skill in the art. For example, a number of histologically normal saliva samples from subjects that are clinically normal (i.e., do not have clinical signs of cancer) are assayed and the mean level of expression for the samples is determined. Likewise, a number of advanced stage cancer saliva samples are assayed and the mean level of expression is determined. Biomarker expression levels of control and cancer saliva samples are compared for a determination of the early diagnosis of cancer. For example, expression levels of one or more biomarkers in a saliva sample can be about two times or greater than the level of expression of those biomarkers in a control saliva sample, but less than the biomarker expression levels of a cancer saliva sample. More specifically, sample biomarker expression levels greater than about 2, 3, 4, 5, 6, 7, 8, 9, 10, 15, or 20 times or more than the level of biomarker expression in the normal control saliva sample, but less than the biomarker expression levels of a cancer saliva sample indicates the early diagnosis cancer in the saliva sample.

Biomarker expression levels may be detected and quantified at the protein level. Methods for detecting proteins or measuring protein levels in biological samples are well known in the art. Many such methods employ antibodies (e.g., monoclonal or polyclonal antibodies) that bind specifically to target proteins. In such assays, an antibody itself or a secondary antibody that binds to it can be detectably labeled. Alternatively, the antibody can be conjugated with biotin, and detectably labeled avidin (a polypeptide that binds to biotin) can be used to detect the presence of the biotinylated antibody. Combinations of these approaches (including “multi-layer sandwich” assays) familiar to those in the art can be used to enhance the sensitivity of the methodologies. Some of these protein measuring assays (e.g., ELISA or Western blot) can be applied to body fluids or to lysates of test cells and others (e.g., immunohistological methods or fluorescence flow cytometry) applied to unlysed tissues or cell suspensions. Methods of measuring the amount of a label depend on the nature of the label and are known in the art, Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-glactosidase), fluorescent moieties or proteins (e.g., fluorescein, rhodamine, phycoerythrin, GFP, or BFP), or luminescent moieties. Other applicable assays include quantitative immunoprecipitation or complement fixation assays.

Saliva samples are collected from control subjects as well as subjects with cancer. Controls can be divided into four categories: (1) healthy control, (2) control+smoker, (3) control+drinker, and (4) control+smoker+drinker. A healthy control is a subject that does not smoke, drink or have any other risk factors associated with the development of head and neck cancer. A control+smoker is a subject that is a smoker, but has not developed head and neck cancer. A control+drinker is a subject that drinks, but has not developed head and neck cancer. A control+smoker+drinker is a subject that smokes and drinks, but has not developed head and neck cancer.

One of the hallmarks and early events of carcinogenesis is tissue invasion and metastasis. Cancer cells can break away from their site or organ of origin to invade surrounding tissue and spread (metastasize) to distant body parts. Mutations of cadherin family members, specially DSG3 promotes this early event. Based on these observations, the inventors postulated that DSG3 level in saliva will increase when a premalignant lesion transforms into clinically detectable HNSCC. The data obtained strongly support this prediction.

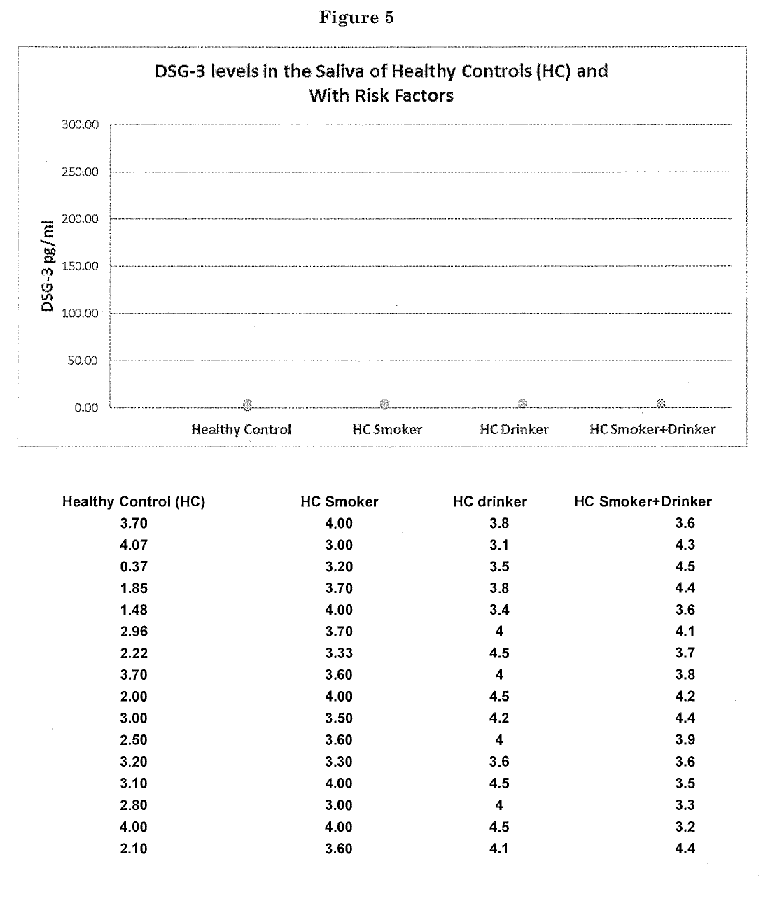

The inventors studied salivary DSG3 level from healthy volunteers, and from those with risk factors for HNSCC, i.e., smoking and alcohol consumption. DSG3 level in saliva among these groups of population were negligible [0.03-4.5 picograms (pg)/ml] (FIG. 5).

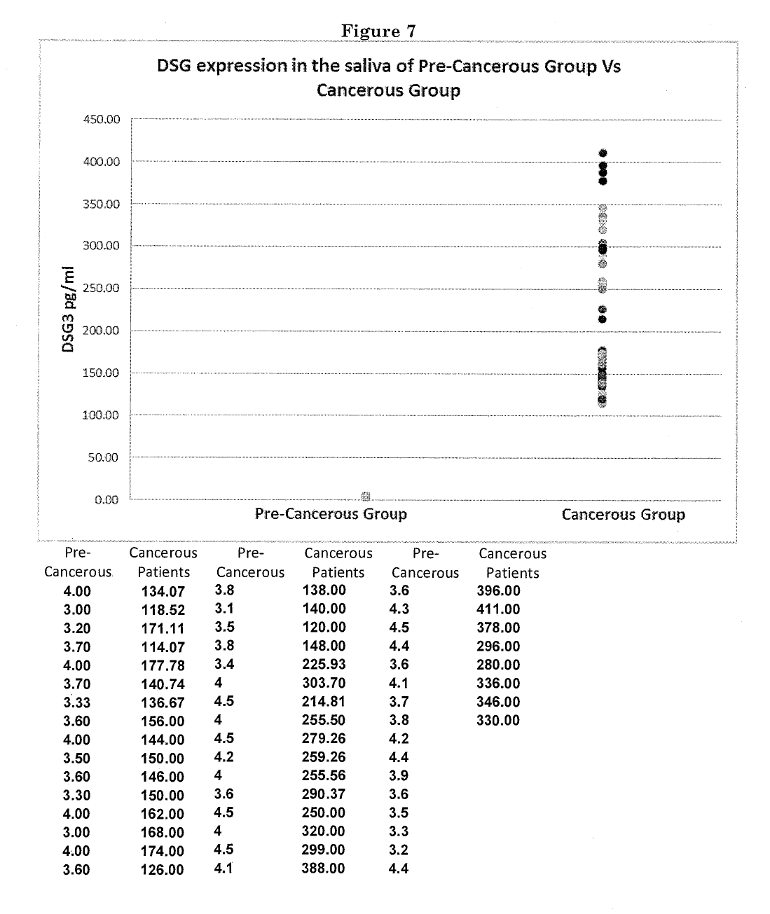

Patients with premalignant lesions also had low levels of DSG3 in saliva (3.2-4.5 pg/ml) (FIG. 7).

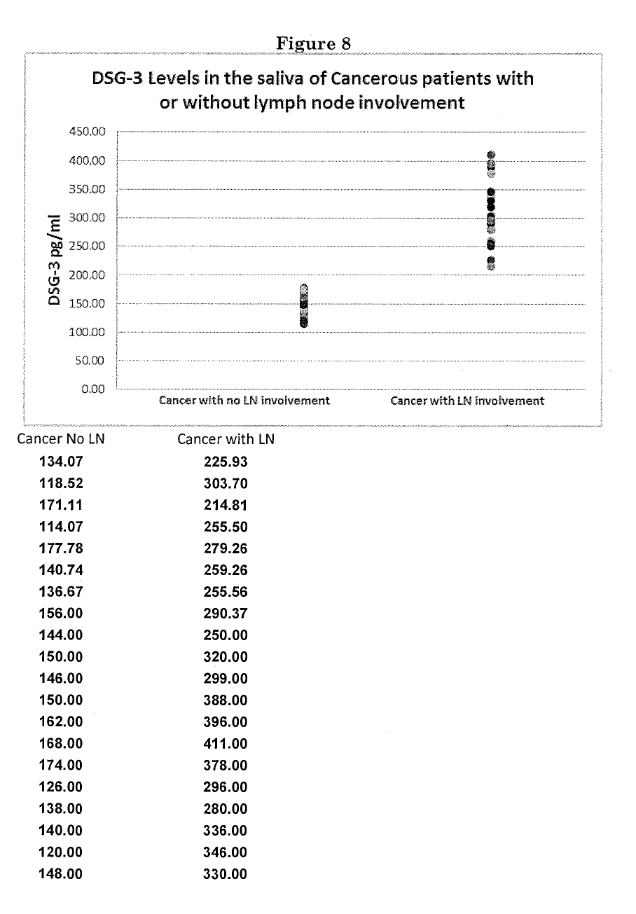

Patients with clinical manifestation of HNSCC had a high level of DSG3 in their saliva (120.00-177.78 pg/ml) (FIG. 8). More interestingly, those patient who DID NOT have metastasis to the cervical lymph nodes had a DSG3 level <200 pg/ml in saliva. However, if cancer metastasized to the cervical lymph nodes, DSG3 in saliva reached much higher level (214.81-411.00 pg/ml) (FIG. 8).

From this study, the inventors conclude:

1) DSG3 can be used as a salivary biomarker to differentiate a premalignant lesion from a squamous cell carcinoma of head and neck.

2) DSG3 can be utilized as a biomarker for cervical lymph node metastasis.

DSG3 biomarker in saliva should help clinicians tremendously as follows:

1) The majority of the patients with oral lesions (benign or pre-malignant) undergo biopsy for the confirmation of diagnosis and mostly, to rule out the possibility of malignancy. The inventors' data shows that it costs approximately $7,000 to perform biopsy by a surgeon and interpret results by a pathologist. Evaluation of DSG3 salivary biomarker should save patients from undergoing unnecessary surgery. This approach should decrease patient morbidity and improve cost-containment.

2) There is usually a dilemma regarding management of cervical lymph nodes for Stage I and II HNSCC since there is no radiologic evidence of cervical lymph node metastasis, although patients may have microscopic metastasis in the lymph node. DSG3 level in saliva has potential to resolve this issue. If the DSG3 level in saliva is >200 pg/ml, a patient should undergo neck dissection with removal of cervical lymph nodes, since there is a high likelihood that the patient will have microscopic metastasis in the lymph nodes.

Cancer saliva samples include: early stage (I & II) head and neck cancer, advanced stage (III & IV) head and neck cancer, and both exophilic and endophilic tongue cancers.

The following protocol for saliva collection was used:
1. The subject was told the time that saliva would be collected (early morning if possible). The subject was asked to refrain from eating, drinking, or oral hygiene procedures for at least half hour prior to the collection.
2. The subject was given drinking water and asked to rinse their mouth out well (without swallowing the water).
3. Five minutes after this oral rinse, the subject was asked to spit around 5 ml of saliva into a 50 cc Falcon tube. The tube was then placed on ice. The subject was reminded not to cough up mucus as saliva was desired, not phlegm.
4. The saliva samples were divided into two tubes. The “Sample 1” tube contained 1 ml of saliva and this tube was used for the protein extraction. The “Sample 2” tube contained approximately 4 ml of saliva which was used for the RNA extraction.
5. “Sample 1”: Centrifuge at 2600 g for 15 minutes at 4° C. If incomplete separation occurred, “Sample 1” was spun for an additional 20 minutes.
6. The supernatant was transferred to a new tube and the following protease inhibitor was added in 1 ml of saliva. 1 microliter of Aprotinin (Stock 10 mg/ml), 3 microliters of Na₃OV₄ (Stock solution 400 mM), 10 microliters of PMSF (Stock solution 10 mg/ml). The solution was mixed well and frozen at −80° C. immediately.
7. “Sample 2”: An equal volume of RNA later stabilization reagent was added to this tube. An aliquot of several tubes was made. These sample tubes were marked and then frozen at ˜80° C. RNAs were extracted from these specimens according to the modified protocol from the manufacturer (RNeasy Mini Kit, Qiagen, Valencia, Calif.).

Saliva was tested for DSG-3 in protein and the RNA level. The inventors discovered that Desmoglein 3 (DSG3) proteins expressed high levels within HNSCC patients. The inventors also demonstrated that the expression of this protein increases as the disease progresses and thus, while not wanting to be bound by theory, these proteins appear to have a direct role in the development of HNSCC. The molecular mechanisms for the progression of head and neck squamous cell carcinoma (HNSCC) cancers are not well understood but are widely believed to involve alcohol, tobacco and deregulation of growth factors leading to development of cancer. The inventors have earlier demonstrated that some of the following proteins such as IL-8, IL-6, VEGF, MMP-9, and TGF beta are elevated in the normal person who may smoke or drink heavily. These proteins are used as tools to develop a prognostic kit in HNSCC. The inventors are able to determine the high risk factors in development of HNSCC among normal people who smoke or drink. The inventors received US patent on IL-8 in 2011 (U.S. Pat. No. 7,910,293, the entire contents of which are incorporated herein by reference in its entirety). DSG-3 is one of seven known desmosomal cadherins that mediate cell-cell adhesion in desmosomes and it is only present in the epithelial cells. The inventors are believed to be the first to detect the expression of DSG-3 from saliva of the oral patient with increased levels of expression correlating with the clinical stage of malignancy, implicating its potential to serve as a diagnostic and prognostic marker. DSG-3 was not detected in the saliva of controls. It was demonstrated that DSG-3 expression was not detected in a normal person who may smoke or drink heavily. DSG-3 can be used in an early prognostic kit that will help to determine how long it will take to develop HNSCC cancer without changing a lifestyle that involves heavy drinking and/or smoking.

The levels of IL-8 and DSG-3 will be detected in the saliva. This test will be more sensitive and specific for the detection of oral cancer at an early stage. Moreover, these proteins will also be an early indicator of the disease status for those individual who are at high risk for developing oral cancer. The inventors have also demonstrated that the combination of DSG-3 and IL-8 will have a compelling increase in efficacy for diagnostics compared to the efficacy of either DSG-3 or IL-8, by itself.

There are at least three main types of treatment head and neck cancer. These include radiation therapy, surgery and chemotherapy. The primary treatments are radiation therapy or surgery, or both combined. Chemotherapy is often used as an additional, or adjuvant, treatment. The optimal combination of these three treatments depends on the site of the cancer and the stage (extent) of the disease.

In general, patients with early-stage head and neck cancers (particularly those limited to the site of origin) are subjected to one treatment—either radiation therapy or surgery. Patients who have more extensive cancers are often treated concurrently with both chemotherapy and radiation therapy. Sometimes, patients are treated with surgery followed by postoperative radiation therapy chemotherapy.

If the plan of treatment is radiation therapy alone for the primary cancer, the neck is also treated with radiation therapy. In addition, a neck dissection to remove involved lymph nodes may be necessary if the amount of disease in the neck nodes is extensive or if the cancer in the neck nodes has not been eliminated completely by the radiation therapy.

Another treatment that might be necessary before or after radiation therapy is surgery. In general, if the surgical removal of the primary tumor is indicated, radiation is given afterward if necessary. Sometimes, however, the cancer is extensive or it is not feasible to completely remove the cancer initially. Radiotherapy is then given first to try to shrink the tumor, and surgery will follow radiotherapy.

Studies indicate that chemotherapy given at the same time as radiation therapy is more effective than if it is given before a course of radiation therapy. Therefore, radiation treatment schedules sometimes include chemotherapy if the stage of the cancer is advanced (advanced stage III or stage IV). Drugs most commonly given in conjunction with radiation therapy are cisplatin (Platinol) and Cetuximab (Erbitux). Additional drugs include but are not limited to fluorouracil (5-FU, Adrucil), carboplatin (Paraplatin), and paclitaxel (Taxol). The chemotherapy may be given in a variety of ways, including a low daily dose, a moderately low weekly dose, or a relatively higher dose every three to four weeks.

One of the following radiation therapy procedures may be used to treat Head and Neck Cancer:

External beam therapy (EBT): a method for delivering a beam of high-energy x-rays or proton beams to the location of the tumor. The beam is generated outside the patient (usually by a linear accelerator x-ray and cyclotron or synchrotron for proton beam) and is targeted at the tumor site. These x-rays can destroy the cancer cells and careful treatment planning allows the surrounding normal tissues to be spared. No radioactive sources are placed inside the patient's body.

Intensity-modulated radiation therapy (IMRT): an advanced mode of high-precision radiotherapy that utilizes computer-controlled x-ray accelerators to deliver precise radiation doses to a malignant tumor or specific areas within the tumor. The radiation dose is designed to conform to the three-dimensional (3-D) shape of the tumor by modulating—or controlling—the intensity of the radiation beam to focus a higher radiation dose to the tumor while minimizing radiation exposure to healthy cells.

Nucleic acid and protein sequences DSG3 are publicly available. For example, GENBANK® Accession Nos.: NM_001944, M76482, AK290367, and BX538327 disclose exemplary nucleic acid sequences that encode human DSG3, and GENBANK® Accession Nos.: NP_001935, AAA60230, BAF83056, and CAD98098 disclose exemplary human DSG-3 amino acid sequences, all of which are incorporated by reference (see e.g., U.S. Patent Application Publication No. 2012/0087892, the entire contents of which are incorporated herein by reference in its entirety).

DSG3 can include a full-length wild-type (or native) sequence, as well as DSG3 allelic variants that retain the ability to be expressed. DSG3 allelic variants have at least 80% sequence identity, for example at least 85%, 90%, 95%, or 98% sequence identity to a publicly available DSG-3 sequence.

NP_001944.2 (SEQ ID NO: 1) is as follows:

| | |
|---|---|
| 1 | aaagcagcag agacgctgca gagggctttt cttagacatc |
| | aactgcagac ggctggcagg |
| 61 | atagaagcag cggctcactt ggactttttc accagggaaa |
| | tcagagacaa tgatggggct |
| 121 | cttccccaga actacagggg ctctggccat cttcgtggtg |
| | gtcatattgg ttcatggaga |
| 181 | attgcgaata gagactaaag gtcaatatga tgaagaagag |
| | atgactatgc aacaagctaa |
| 241 | aagaaggcaa aaacgtgaat gggtgaaatt tgccaaaccc |
| | tgcagagaag gagaagataa |
| 301 | ctcaaaaaga aacccaattg ccaagattac ttcagattac |
| | caagcaaccc agaaaatcac |
| 361 | ctaccgaatc tctggagtgg gaatcgatca gccgcctttt |
| | ggaatctttg ttgttgacaa |
| 421 | aaacactgga gatattaaca taacagctat agtcgaccgg |
| | gaggaaactc caagcttcct |
| 481 | gatcacatgt cgggctctaa atgcccaagg actagatgta |
| | gagaaaccac ttatactaac |
| 541 | ggttaaaatt ttggatatta atgataatcc tccagtattt |
| | tcacaacaaa ttttcatggg |
| 601 | tgaaattgaa gaaaatagtg cctcaaactc actggtgatg |
| | atactaaatg ccacagatgc |
| 661 | agatgaacca aaccacttga attctaaaat tgccttcaaa |
| | attgtctctc aggaaccagc |
| 721 | aggcacaccc atgttcctcc taagcagaaa cactggggaa |
| | gtccgtactt tgaccaattc |
| 781 | tcttgaccga gagcaagcta gcagctatcg tctggttgtg |
| | agtggtgcag acaaagatgg |
| 841 | agaaggacta tcaactcaat gtgaatgtaa tattaaagtg |
| | aaagatgtca acgataactt |
| 901 | cccaatgttt agagactctc agtattcagc acgtattgaa |
| | gaaaatattt taagttctga |
| 961 | attacttcga tttcaagtaa cagatttgga tgaagagtac |
| | acagataatt ggcttgcagt |
| 1021 | atatttcttt acctctggga atgaaggaaa ttggtttgaa |
| | atacaaactg atcctagaac |
| 1081 | taatgaaggc atcctgaaag tggtgaaggc tctagattat |
| | gaacaactac aaagcgtgaa |
| 1141 | acttagtatt gctgtcaaaa acaaagctga atttcaccaa |
| | tcagttatct ctcgataccg |
| 1201 | agttcagtca accccagtca caattcaggt aataaatgta |
| | agagaaggaa ttgcattccg |
| 1261 | tcctgcttcc aagacattta ctgtgcaaaa aggcataagt |
| | agcaaaaaat tggtggatta |
| 1321 | tatcctggga acatatcaag ccatcgatga ggacactaac |
| | aaagctgcct caaatgtcaa |
| 1381 | atatgtcatg ggacgtaacg atggtggata cctaatgatt |
| | gattcaaaaa ctgctgaaat |
| 1441 | caaatttgtc aaaaatatga accgagattc tactttcata |
| | gttaacaaaa caatcacagc |
| 1501 | tgaggttctg gccatagatg aatacacggg taaaacttct |
| | acaggcacgg tatatgttag |
| 1561 | agtacccgat ttcaatgaca attgtccaac agctgtcctc |
| | gaaaaagatg cagtttgcag |
| 1621 | ttcttcacct tccgtggttg tctccgctag aacactgaat |
| | aatagataca ctggccccta |
| 1681 | tacatttgca ctggaagatc aacctgtaaa gttgcctgcc |
| | gtatggagta tcacaaccct |
| 1741 | caatgctacc tcggccctcc tcagagccca ggaacagata |
| | cctcctggag tataccacat |
| 1801 | ctccctggta cttacagaca gtcagaacaa tcggtgtgag |
| | atgccacgca gcttgacact |
| 1861 | ggaagtctgt cagtgtgaca acaggggcat ctgtggaact |
| | tcttacccaa ccacaagccc |
| 1921 | tgggaccagg tatggcaggc cgcactcagg gaggctgggg |
| | cctgccgcca tcggcctgct |
| 1981 | gctccttggt ctcctgctgc tgctgttggc cccccttctg |
| | ctgttgacct gtgactgtgg |
| 2041 | ggcaggttct actgggggag tgacaggtgg ttttatccca |
| | gttcctgatg gctcagaagg |
| 2101 | aacaattcat cagtggggaa ttgaaggagc ccatcctgaa |
| | gacaaggaaa tcacaaatat |
| 2161 | ttgtgtgcct cctgtaacag ccaatggagc cgatttcatg |
| | gaaagttctg aagtttgtac |
| 2221 | aaatacgtat gccagaggca cagcggtgga aggcacttca |
| | ggaatggaaa tgaccactaa |
| 2281 | gcttggagca gccactgaat ctggaggtgc tgcaggcttt |
| | gcaacaggga cagtgtcagg |
| 2341 | agctgcttca ggattcggag cagccactgg agttggcatc |
| | tgttcctcag ggcagtctgg |
| 2401 | aaccatgaga acaaggcatt ccactggagg aaccaataag |
| | gactacgctg atggggcgat |
| 2461 | aagcatgaat tttctggact cctacttttc tcagaaagca |
| | tttgcctgtg cggaggaaga |
| 2521 | cgatggccag gaagcaaatg actgcttgtt gatctatgat |
| | aatgaaggcg cagatgccac |
| 2581 | tggttctcct gtgggctccg tgggttgttg cagttttatt |
| | gctgatgacc tggatgacag |
| 2641 | cttcttggac tcacttggac ccaaatttaa aaaacttgca |
| | gagataagcc ttggtgttga |
| 2701 | tggtgaaggc aaagaagttc agccaccctc taaagacagc |
| | ggttatggga ttgaatcctg |
| 2761 | tggccatccc atagaagtcc agcagacagg atttgttaag |
| | tgccagactt tgtcaggaag |
| 2821 | tcaaggagct tctgctttgt ccacctctgg gtctgtccag |
| | ccagctgttt ccatccctga |
| 2881 | ccctctgcag catggtaact atttagtaac ggagacttac |
| | tcggcttctg gttccctcgt |
| 2941 | gcaaccttcc actgcaggct ttgatccact tctcacacaa |
| | aatgtgatag tgacagaaag |
| 3001 | ggtgatctgt cccatttcca gtgttcctgg caacctagct |
| | ggcccaacgc agctacgagg |
| 3061 | gtcacatact atgctctgta cagaggatcc ttgctcccgt |
| | ctaatatgac cagaatgagc |
| 3121 | tggaatacca cactgaccaa atctggatct ttggactaaa |
| | gtattcaaaa tagcatagca |
| 3181 | aagctcactg tattgggcta ataatttggc acttattagc |
| | ttctctcata aactgatcac |
| 3241 | gattataaat taaatgtttg ggttcatacc ccaaaagcaa |
| | tatgttgtca ctcctaattc |
| 3301 | tcaagtacta ttcaaattgt agtaaatctt aaagtttttc |
| | aaaaccctaa aatcatattc |
| 3361 | gccaggaaat tttcctaaac attcttaagc ttctattttt |
| | cccctgccaa aggaaggtgt |
| 3421 | ttatcatttt aaaatgcaat gtgatttagt ggattaagca |
| | ggagcgctgg ttcttgtctc |
| 3481 | cattgccttt tcttatatca ttgataatga tgtaagaatc |
| | acaaggggcc gggcgcggtg |
| 3541 | gctcacgcct gtaatcccag cactttggga ggccgaggca |
| | ggtggatcat gaggtcagga |
| 3601 | gatcgagacc atcctggcta acaaggtgaa accccgtctc |
| | tactaaaaat acaaaaaatt |
| 3661 | agccgggcgc agtggcgggc gcctgtagtc ccagctactc |
| | gggaggctga ggcaggagaa |
| 3721 | tggcatgaac ccgggaagcg gagcttgcag tgagccgaga |
| | ttgcgccact gcagtccgca |
| 3781 | gtccggcctg ggcgacagag cgagactccg tctcaaaaaa |
| | aaaaaaaaaa aaagaatcac |
| 3841 | aaggtatttg ctaaagcatt ttgagctgct tggaaaaagg |
| | gaagtagttg cagtagagtt |
| 3901 | tcttccatct tcttggtgct gggaagccat atatgtgtct |
| | tttactcaag ctaaggggta |
| 3961 | taagcttatg tgttgaattt gctacatcta tatttcacat |
| | attctcacaa taagagaatt |
| 4021 | ttgaaataga aatatcatag aacatttaag aaagtttagt |
| | ataaataata ttttgtgtgt |
| 4081 | tttaatccct ttgaagggat ctatccaaag aaaatatttt |
| | acactgagct ccttcctaca |
| 4141 | cgtctcagta acagatcctg tgttagtctt tgaaaatagc |
| | tcatttttta aatgtcagtg |
| 4201 | agtagatgta gcatacatat gatgtataat gacgtgtatt |
| | atgttaacaa tgtctgcaga |
| 4261 | ttttgtagga atacaaaaca tggccttttt tataagcaaa |
| | acgggccaat gactagaata |
| 4321 | acacataggg caatctgtga atatgtatta taagcagcat |
| | tccagaaaag tagttggtga |
| 4381 | aataattttc aagtcaaaaa gggatatgga aagggaatta |
| | tgagtaacct ctatttttta |
| 4441 | agccttgctt ttaaattaaa cagctacagc catttaagcc |
| | ttgaggataa taaagcttga |
| 4501 | gagtaataat gttaggttag caaaggttta gatgtatcac |
| | ttcatgcatg ctaccatgat |
| 4561 | agtaatgcag ctcttcgagt catttctggt cattcaagat |
| | attcaccctt ttgcccatag |
| 4621 | aaagcaccct acctcacctg cttactgaca ttgtcttagc |
| | tgatcacaag atcattatca |
| 4681 | gcctccatta ttccttactg tatataaaat acagagtttt |
| | atattttcct ttcttcgttt |
| 4741 | ttcaccatat tcaaaaccta aatttgtttt tgcagatgga |
| | atgcaaagta atcaagtgtt |
| 4801 | tgtgctttca cctagaaggg tgtggtcctg aaggaaagag |
| | gtcccctaaa tatcccccac |
| 4861 | cctggtgctc ctccctctcc ctggtaccct gactaccagg |
| | aagtcaggtg ctagagcagc |
| 4921 | tggagaagtg caggcagcct gtgcttccac agatgggggt |
| | gctgctgcaa caaggctttc |
| 4981 | aatgtgccca tcttaggtgg gagaagctag atcctgtgca |
| | gcagcctggt aagtcctgag |
| 5041 | gaggttccat tgctcttcct gctgctgtcc tttgcttctc |
| | aacggtggct cgctctacag |
| 5101 | tctagagcac atgcagctaa cttgtgcctc tgcttatgca |
| | tgagggttaa attaacaacc |
| 5161 | ataaccttca tttgaagttc aaaggtgtat tcaggatcct |
| | caaagcattt taaccttgcc |
| 5221 | gcttaaaacc caatttaccg tgaaatggga attttgctgc |
| | attgttaaac tgtagtggaa |
| 5281 | accatgctat agtaataaag gttatataag agagaaattg |
| | aaattaaatg tgtttttaaa |
| 5341 | tttcaaaaaa aaatcaatct ttaggatgac ttaaaaattg |
| | atttgccatg taaaatgtat |
| 5401 | ctgcattttt tacacaaaac ttgttttaag cataaaattt |
| | taaaactgta ctacttgatg |
| 5461 | tattatacat tttgaaccat atgtattaaa ccataaacag |
| | tataatgttg ttataataaa |
| 5521 | acaggcaata aatttataaa taaaagctga aaaaaaaaaa a |

NP_001935.2 (SEQ ID NO: 2) is as follows:

| | |
|---|---|
| 1 | mmglfprttg alaifvvvil vhgelrietk gqydeeemtm |
| | qqakrrqkre wvkfakpere |
| 61 | gednskrnpi akitsdyqat qkityrisgv gidqppfgif |
| | vvdkntgdin itaivdreet |
| 121 | psflitcral naqgldvekp liltvkildi ndnppvfsqq |
| | ifmgeieens asnslvmiln |
| 181 | atdadepnhl nskiafkivs qepagtpmfl lsrntgevrt |
| | ltnsldreqa ssyrlvvsga |
| 241 | dkdgeglstq cecnikvkdv ndnfpmfrds qysarieeni |
| | lssellrfqv tdldeeytdn |
| 301 | wlavyfftsg negnwfeiqt dprtnegilk vvkaldyeql |
| | qsvklsiavk nkaefhqsvi |
| 361 | sryrvqstpv tiqvinvreg iafrpasktf tvqkgisskk |
| | lvdyilgtyq aidedtnkaa |
| 421 | snvkyvmgrn dggylmidsk taeikfvknm nrdstfivnk |
| | titaevlaid eytgktstgt |
| 481 | vyvrvpdfnd ncptavlekd avcssspsvv vsartlnnry |
| | tgpytfaled qpvklpavws |
| 541 | ittlnatsal lraqeqippg vyhislvltd sqnnrcempr |
| | sltlevcqcd nrgicgtsyp |
| 601 | ttspgtrygr phsgrlgpaa igllllglll lllapllllt |
| | cdcgagstgg vtggfipvpd |
| 661 | gsegtihqwg iegahpedke itnicvppvt angadfmess |
| | evctntyarg tavegtsgme |
| 721 | mttklgaate sggaagfatg tvsgaasgfg aatgvgicss |
| | gqsgtmrtrh stggtnkdya |
| 781 | dgaismnfld syfsqkafac aeeddgqean dclliydneg |
| | adatgspvgs vgccsfiadd |
| 841 | lddsfldslg pkfkklaeis lgvdgegkev qppskdsgyg |
| | iescghpiev qqtgfvkcqt |
| 901 | lsgsqgasal stsgsvqpav sipdplqhgn ylvtetysas |
| | gslvqpstag fdplltqnvi |
| 961 | vtervicpis svpgnlagpt qlrgshtmlc tedpcsrli |

A database generated from the methods provided herein and the analyses described above can be included in, or associated with, a computer system for the early detection of head and neck cancer. The database can include a plurality of digitally-encoded “reference” (or “control”) profiles. Profiles can be included in the database for consecutive or simultaneous comparison to a subject profile. The computer system can include a server containing a computer-executable code for receiving a profile of a subject and identifying from the database a matching reference profile that is diagnostically relevant to the subject profile.

Thus, the various techniques, methods, and aspects of the invention described above can be implemented in part or in whole using computer-based systems and methods. Additionally, computer-based systems and methods can be used to augment or enhance the functionality described above, increase the speed at which the functions can be performed, and provide additional features and aspects as a part of or in addition to those of the invention described elsewhere in this document. Various computer-based systems, methods and implementations in accordance with the above-described technology are presented below.

A processor-based system can include a main memory, preferably random access memory (RAM), and can also include a secondary memory. The secondary memory can include, for example, a hard disk drive and/or a removable storage drive, representing a floppy disk drive, a magnetic tape drive, an optical disk drive, etc. The removable storage drive reads from and/or writes to a removable storage medium. Removable storage medium refers to a floppy disk, magnetic tape, optical disk, and the like, which is read by and written to by a removable storage drive. As will be appreciated, the removable storage medium can comprise computer software and/or data.

In alternative embodiments, the secondary memory may include other similar means for allowing computer programs or other instructions to be loaded into a computer system. Such means can include, for example, a removable storage unit and an interface. Examples of such can include a program cartridge and cartridge interface (such as the found in video game devices), a removable memory chip (such as an EPROM or PROM) and associated socket, and other removable storage units and interfaces, which allow software and data to be transferred from the removable storage unit to the computer system.

The computer system can also include a communications interface. Communications interfaces allow software and data to be transferred between computer system and external devices. Examples of communications interfaces can include a modem, a network interface (such as, for example, an Ethernet card), a communications port, a PCMCIA slot and card, and the like. Software and data transferred via a communications interface are in the form of signals, which can be electronic, electromagnetic, optical or other signals capable of being received by a communications interface. These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other communications channels.

The terms “computer program medium” and “computer usable medium” are used to refer generally to media such as a removable storage device, a disk capable of installation in a disk drive, and signals on a channel. These computer program products are means for providing software or program instructions to a computer system.

Computer programs (also called computer control logic) are stored in main memory and/or secondary memory. Computer programs can also be received via a communications interface. Such computer programs, when executed, enable the computer system to perform the features of the invention as discussed herein. In particular, the computer programs, when executed, enable the processor to perform the features of the invention. Accordingly, such computer programs represent controllers of the computer system.

In an embodiment where the elements are implemented using software, the software may be stored in, or transmitted via, a computer program product and loaded into a computer system using a removable storage drive, hard drive or communications interface. The control logic (software), when executed by the processor, causes the processor to perform the functions of the invention as described herein.

In another embodiment, the elements are implemented primarily in hardware using, for example, hardware components such as PALs, application specific integrated circuits (ASICs) or other hardware components. Implementation of a hardware state machine so as to perform the functions described herein will be apparent to person skilled in the relevant art(s). In yet another embodiment, elements are implanted using a combination of both hardware and software.

In another embodiment, the computer-based methods can be accessed or implemented over the World Wide Web by providing access via a Web Page to the methods of the invention. Accordingly, the Web Page is identified by a Universal Resource Locator (URL). The URL denotes both the server machine and the particular file or page on that machine. In this embodiment, it is envisioned that a consumer or client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol (“HTTP”)). The selected page is then displayed to the user on the client's display screen. The client may then cause the server containing a computer program of the invention to launch an application to, for example, perform an analysis according to the invention.

### REFERENCES

All references cited herein, including those below are hereby incorporated by reference herein in their entirety.
1. U.S. Pat. No. 7,910,293.
2. U.S. Patent Application Publication No. 2010/0099102.
3. U.S. Patent Application Publication No. 2012/0087892.

What is claimed is:

1. A method for the early detection of head and neck cancer comprising:
obtaining a saliva sample from a subject;
determining the expression level of Desmoglien 3 (DSG3) in said saliva sample; and
detecting head and neck cancer by the expression level of DSG3.

2. The method of claim 1, wherein the step of detecting head and neck cancer comprises comparing the expression level of DSG3 with the average expression levels of DSG3 from control saliva samples and advanced stage cancer saliva samples, wherein said control saliva samples are from subjects selected from the group consisting of a) smokers, b) drinkers, c) smokers and drinkers, and d) non-smokers and non-drinkers, all of whom had not yet developed head and neck cancer, and wherein a determination that the level of expression of DSG3 in said saliva sample is greater than the average level of expression of DSG3 in said control saliva samples, but is less than the average DSG3 expression level of the advanced stage cancer saliva samples is indicative of the early detection of head and neck cancer in the subject.

3. The method according to claim 2, wherein said head and neck cancer comprises lip, oral cavity, tongue, throat, nasal cavity, paranasal sinuses, pharynx, larynx, salivary glands and cervical lymph nodes of the neck.

4. The method according to claim 2, wherein DSG3 expression levels of said saliva samples are at least two times greater than the DSG3 expression levels of the control saliva samples.

5. The method according to claim 2, wherein DSG3 expression levels of said saliva samples are at least five times greater than the DSG3 expression levels of the control saliva samples.

6. A method for the early detection of head and neck cancer comprising:
obtaining a saliva sample from a subject;
determining the expression level of Desmoglien 3 (DSG3) and one or more other biomarkers selected from the group consisting of IL-1, IL-6, IL-8, VEGF, TGFβ, TNFα, MMP-7, plasminogen activated (PA), uPA, IGF, and INF-2 proteins in said saliva sample; and
detecting head and neck cancer by the expression level of DSG3 and one or more other biomarkers selected from the group consisting of IL-1, IL-6, IL-8, VEGF, TGFβ, TNFα, MMP-7, plasminogen activated (PA), uPA, IGF, and INF-2 proteins.

7. The method of claim 6, wherein the step of detecting head and neck cancer comprises comparing the expression level of DSG3 and said one or more other biomarkers with the average expression levels of DSG3 and said one or more other biomarkers from control saliva samples and advanced stage cancer saliva samples, wherein said control saliva samples are from subjects selected from the group consisting of a) smokers, b) drinkers, c) smokers and drinkers, and d) non-smokers and non-drinkers, all of whom had not yet developed head and neck cancer, and wherein a determination that the level of expression of DSG3 and said one or more other biomarkers in said saliva sample is greater than the average level of expression of DSG3 and said one or more other biomarkers in said control saliva samples, but is less than the average DSG3 and said one or more other biomarkers expression level of the advanced stage cancer saliva samples is indicative of the early detection of head and neck cancer in the subject.

8. The method according to claim 7, wherein said head and neck cancer comprises lip, oral cavity, tongue, throat, nasal cavity, paranasal sinuses, pharynx, larynx, salivary glands and cervical lymph nodes of the neck.

9. The method according to claim 7, wherein said one or more other biomarkers comprise IL-8.

10. The method according to claim 9, wherein DSG3 and IL-8 expression levels of said saliva samples are at least two times greater than the DSG3 and IL-8 expression levels of the control saliva samples.

11. The method according to claim 9, wherein DSG3 and IL-8 expression levels of said saliva samples are at least five times greater than the DSG3 and IL-8 expression levels of the control saliva samples.

12. A method for treating head and neck cancer comprising performing one or more procedures selected from the group consisting of radiation therapy, surgery and chemotherapy on a subject, wherein the procedure is performed when early detection of head and neck cancer is detected by the method of claim 2.

13. The method according to claim 12, wherein said head and neck cancer comprises lip, oral cavity, tongue, throat, nasal cavity, paranasal sinuses, pharynx, larynx, salivary glands and cervical lymph nodes of the neck.

14. The method according to claim 12, wherein DSG3 expression levels of said saliva samples are at least two times greater than the DSG3 expression levels of the control saliva samples.

15. The method according to claim 12, wherein DSG3 expression levels of said saliva samples are at least five times greater than the DSG3 expression levels of the control saliva samples.

16. A method for treating head and neck cancer comprising performing one or more procedures selected from the group consisting of radiation therapy, surgery and chemotherapy on a subject, wherein the procedure is performed when early detection of head and neck cancer is detected by the method of claim 6.

17. The method according to claim 16, wherein said head and neck cancer comprises lip, oral cavity, tongue, throat, nasal cavity, paranasal sinuses, pharynx, larynx, salivary glands and cervical lymph nodes of the neck.

18. The method according to claim 16, wherein said one or more other biomarkers comprise IL-8.

19. The method according to claim 18, wherein DSG3 and IL-8 expression levels of said saliva samples are at least two times greater than the DSG3 and IL-8 expression levels of the control saliva samples.

20. The method according to claim 18, wherein DSG3 and IL-8 expression levels of said saliva samples are at least five times greater than the DSG3 and IL-8 expression levels of the control saliva samples.
